SEQUENCE LISTING (2 sequences)

SEQ ID NO: 1 - DNA, 36 bp, Artificial Sequence
  Synthetic oligonucleotide comprising EcoR1 cleavage site.
agtaacacggtcatcgaattccagccttagaagctt

SEQ ID NO: 2 - DNA, 36 bp, Artificial Sequence
  Synthetic oligonucleotide comprising EcoR1 cleavage site.
tcattgtgccagtagcttaaggtcggaatcttcgaa

(19) United States
(12) Patent Application Publication
(10) Pub. No.: US 2004/0038316 A1
(43) Pub. Date: Feb. 26, 2004

(54) Cell separation device and methods for use

(76) Inventors: Robert Kaiser, Kenmore, WA (US); Carl Weissman, Seattle, WA (US); Irving L. Weissman, Redwood City, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/648,142

(22) Filed: Aug. 25, 2003

Related U.S. Application Data
(63) Continuation of application No. 09/568,646, filed on May 10, 2000, now abandoned.
(60) Provisional application No. 60/133,342, filed on May 10, 1999.

(51) Int. Cl. G01N 33/53

(52) U.S. Cl. 435/7.2; 435/6

(57) ABSTRACT

An apparatus and methods are provided for the separation of a subset from a complex mixture of particles, e.g. cells, virus, organelles, etc., wherein the particles have a binding moiety present on an accessible, usually external, surface. The apparatus comprises a capillary tube or array of capillary tubes having bound to the luminal surface a capture system comprising a cleavable linkage and a selective ligand. The capture system provides selectivity both in binding particles, through the ligand, and in the release of particles, through the cleavable linker. Each capillary may be divided linearly into zones, where in each zone the composition of the capture system varies, particularly in the ligand specificity. In one embodiment of the invention, each zone comprises a ligand specific for a different positive or negative selection marker on the desired particles.

Cell separation is a rapidly growing area of biomedical and clinical development. Improved methods of separating a desired cell subset from a complex population permit the study and use of cells that have relatively uniform and defined characteristics. Cell separation is widely used in research, e.g. to determine the effect of a drug or treatment on a targeted cell population; investigation of biological pathways; isolation of transformed or otherwise modified cell populations; etc. Present clinical uses include the isolation of hematopoietic stem cells for reconstitution of blood cells, particularly in combination with ablative chemo- and radiation therapy.

Cell separation is achieved by targeting molecules on the cell surface with specific affinity ligands in order to achieve selective, reversible attachment of the target cell population to a solid phase. In a subsequent step, the nonspecifically adsorbed cells are removed by washing, followed by the release of target cells. The specific affinity ligands may be antibodies, lectins, receptor ligands, or other ligands that bind proteins, hormones, carbohydrates, or other molecules with biological activity.

Clinical uses requires a sterile, closed apparatus in which large quantities of viable, specific cell populations can be obtained rapidly from crude cell suspensions or directly from blood. Several systems are currently available for sterile sorting. Flow cytometry in various formats is widely used, and can provide for highly purified cell populations. However, it suffers from being expensive, requiring skilled operators, and being hard to scale up.

Alternatives to flow cytometry include variations of column chromatography, where the column may comprise iron particles; antibodies immobilized on particles: immobilized lectins, and the like. For example, antibodies attached to magnetic particles have been used to separate hematopoietic progenitor cells, where the antibodies are bound to the starting cell population, then separated on a column of iron beads.

Various substrates exist for use with columns to separate the target particle from the sample fluid. Generally, the type of substrate selected for performing the separation will determine how the target particles are separated from the sample fluid. The substrate is selected so that the desired particles have different binding characteristics to the substrate than the remaining components of the sample. An example of a column type apparatus for cell separation may be found in U.S. Pat. No. 5,240,856, issued Aug. 31, 1993, where the cells bind to a matrix within the column. In one version, the column is designed to be pliable, to facilitate removal of bound cells (U.S. Pat. No. 5,695,989, issued Dec. 9, 1997). U.S. Pat. No. 5,672,481, issued Sep. 30, 1997 describes an apparatus for separation in a closed sterile field, where a single vessel is used for collection and concentration and transfer. U.S. Pat. No. 5,763,194 describes a cell separation device comprised of an array of semi-permeable hollow fibers, to which a ligand is attached to the inner surface.

Improved methods of separating cells have a great utility in the many medical and biological fields that require purified populations. Many biological techniques such as are employed in biotechnology, microbiology, clinical diagnostics and treatment, in vitro fertilization, hematology and pathology, require such processes as identification, separation, culturing, or manipulation of a target cell or particle, e.g. cell subsets, platelets, bacteria, virus particles, etc.

Relevant Literature

The use of releasable linkers with superparamagnetic particles is described in International Patent Application no. WO 96/31776. U.S. Pat. No. 5,849,878 describes the use of double stranded DNAs as chemically and spatially defined cross-linkers.

Priest, U.S. Pat. No. 5,391,723, discloses conjugates of oligonucleotides. Also of interest is Amankwa and Kuhr (1992) Analytical Chemistry 64:1610-1613.

### SUMMARY OF THE INVENTION

An apparatus and methods are provided for the separation of a subset from a complex mixture of particles, e.g. cells, virus, organelles, etc., wherein the particles have a binding moiety present on an accessible, usually external, surface. The apparatus comprises a capillary tube or array of capillary tubes having bound to the luminal surface a capture system of the form:

S-Q-(X)ₙ-CL-(Y)ₘ-L

where S is the luminal surface of the capillary tubing; Q is a chemical linkage between the surface and X; X is a linker or affinity reagent; CL is a cleavable linkage; Y is a linker or affinity reagent; and L is a selective ligand capable of specifically binding a moiety present on the desired particle subset. Independently, n and m are chosen to be 1 or 0. Preferred cleavable linkers are compounds that are not ordinarily present on an accessible surface of the particle, e.g. nucleic acid sequences.

Each capillary may be divided linearly into zones, where in each zone the composition of the capture system varies, particularly in the ligand specificity. In one embodiment of the invention, each zone comprises a ligand specific for a different positive or negative selection marker on the desired particles.

The targeted particles are put in contact with the ligand on the luminal surface by flow along the length of the tubing, usually at low flow rates that minimize shear stress. Particles that are not bound are washed through the capillary. The particles bound to the capture ligand are unbound from the surface by cleavage of the cleavable linker with a cleavage reagent. Preferred cleavage reagents are enzymes that are reactive under physiological conditions, so as to minimize deleterious effects of the cleavage reaction on the target particles. The unbound particles are then washed from the capillary surface by flow rates with increased shear stress, optionally coupled with agitation or other physical means.

### BRIEF DESCRIPTION OF THE DRAWINGS

FIG. 1 shows the capillary tubing of the invention, and its division into zones. FIG. 1B illustrates the luminal binding surface of the capillary tubing.

FIG. 2A depicts an array of capillary tubes, and FIG. 2B an apparatus comprising a capillary array, baffles and filters; and dispensing and collection containers.

FIG. 3 depicts a helical arrangement of a capillary array.

FIG. 4 depicts an exemplary zonal capillary system.

### DESCRIPTION OF THE SPECIFIC EMBODIMENTS

Improved biological particle separation apparatus and separation procedures are provided. The separation apparatus comprises a capillary tube or an array of capillary tubes, having bound to their luminal surface a capture system of the form:

S-Q-(X)ₙ—CL-(Y)ₘ-L

where S is the luminal surface of the capillary tubing; Q is a chemical linkage between the surface and X; X is a chemical bond, linker or affinity reagent; CL is a cleavable linkage; Y is a chemical bond, linker of affinity reagent; and L is a ligand capable of specifically binding a moiety present on the desired particle subset.

To separate a targeted particle subset from a complex mixture, the mixture is contacted with the lumen of the capillaries, flowing by gravity, capillary flow, peristaltic pump, etc., usually at low flow rates that minimize shear stress. The targeted particles are bound to the ligand moiety. Particles that are not bound flow through the capillary. The bound particles are released from the surface by cleavage of the cleavable linker. The targeted particles are then washed from the capillary surface by flow rates with increased shear stress, optionally coupled with agitation or other physical means. Where selection zones are present, the released cells are then available for selection in a lower zone of the apparatus, where the selection process can be repeated with a different binding specificity.

Any biological particle comprising a binding moiety present on an accessible surface may be separated by the methods of the present invention. The binding moiety is used to distinguish the targeted particle from other particles present in a complex mixture. The complex mixture may comprise similar particles, e.g. a population of cells; dissimilar particles, e.g. a virus present in a cell population; particles in a suspension, e.g. a colloidal suspension; etc. Particles of interest include cells, organelles, viruses, and the like. Of particular interest are cells from various systems and organs, including multipotential cells such as stem or progenitor cells, including neural crest stem cells, hematopoietic stem cells, embryonic stem cells, mesenchymal stem cells, central nervous system stem cells, etc.

### Definitions

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

Capture system: The capture system is a binding structure that provides selectivity in both binding and release of a targeted particle. The capture system is attached to the luminal surface of a capillary tube, generally coating the capillary tube. In general, the number of particles that can be captured per unit area of the luminal surface will depend on the size of the particle to be captured, the size of the ligand L, the affinity between L and its target binding moiety on the particle surface, and the flow rate of the particles in the capillary. Using optimal flow conditions and a ligand with sufficiently high affinity for the target receptor, generally at least about 10⁻⁴ M affinity, usually at least about 10⁻⁵ M, and preferably at least about 10⁻⁶ M affinity, the number of particles captured per unit area of the luminal surface will be dependent on the relative sizes of the ligand and the particle. Ligands with reduced affinity may be used if there is sufficient avidity in combination with neighboring ligands.

In most applications of this invention, the particle size will be considerably larger than the ligand size. This is the case, for example, when the particle is a human cell (average diameter ˜10⁻²⁵ microns) and the ligand is an antibody (average diameter ˜100-200 angstroms). In general, effects such as steric interference, multiple ligand-receptor interactions between the luminal surface and the particle surface, and non-specific binding of contaminating species in the particle suspension to the luminal surface will reduce the number of particles that can actually be captured per unit area of the luminal surface to about 1-10% of the maximum expected by simple area calculations.

The capture system includes the components:

S-Q-(X)ₙ—CL-(Y)ₘ-L

as previously defined, where n and m are, independently, 1 or 0. The individual components are defined in more detail below. The capture system provides selectivity in particle separation both through the ligand, which specifically binds to a moiety present on an accessible particle surface, and through the cleavable linker, which is stable under normal flow conditions, but which is cleaved in the presence of a specific cleavage reagent. In this way, a targeted particle binds to the capture system when the two are brought into contact, allowing undesirable particles, cells, etc. to be washed away. The particle is then released from the capillary surface by contacting the capture system with a cleavage reagent.

In one embodiment of the invention, either X, or Y, or both, are present as an affinity reagent, so that the selective reagents L and CL can be introduced in a “cassette” form. For example, the capillary tubing may be functionalized along its length with Q; and then with an affinity reagent, such as biotin, a hapten, etc. The capillary may then be stored, and used as needed for various cleavable linkers and selective ligands. A “cassette” of the cleavable linker, alone or in combination with a selective ligand, is attached to a binding partner for X, e.g. avidin, streptavidin, antibody, etc. On contact with X, the CL or CL-L group is then bound to the capillary wall. As a variation on this method, the CL-L group may be pre-bound to the targeted particles.

Positive selection: allows the direct selection and recovery of a specific particle population from a heterogeneous group. It allows the selective enrichment and purification of targeted cells or other biological particles.

Negative selection: involves the elimination of specific cell types from a heterogeneous population. Negative selection techniques have a limitation in that although specific component types can be removed, the remaining components do not necessarily provide a pure population.

Capillary surface (S): The capillary tubing used in the present invention may be those conventionally known and used in the art, having a length of from about 10 cm to 10 m in length, with a diameter ranging from at least about 50 microns to not more than about 500 microns. The capillary walls may be of a flexible or rigid substance, including glass, fused silica, quartz, polystyrene, polyethylene/polypropylene, polycarbonate, polymethylmethacrylate, etc.

Linkage (Q): is the molecular linkage between the luminal capillary surface and species X, when present; or to CL. Q is a linker chosen to be stable under standard operating conditions for the separation procedure, which are generally those that maintain the biological integrity of the sample. Q will provide an attachment to the capillary surface and to CL or X.

It is possible to passively adsorb, in a non-covalent fashion, certain of species X, e.g. proteins and the like, to the luminal surface of the capillary in order to by-pass the need for Q. Such procedures are well known by those skilled in the art, and consist of contacting a solution of X with the surface for a sufficiently long period of time and then washing away unbound X. While the simplicity of this approach is attractive, in general the surface coating will be too unstable for prolonged use to ensure high-purity separations and the choice of X will be quite limited.

To improve the stability of the coating and provide for a greater number of options, the luminal surface is functionalized with Q to facilitate attachment of X or CL. Modes of surface functionalization include silanization of glass-like surfaces by 3-aminopropyltriethoxysilane, 3-mercaptopropyltrimethoxysilane, 3-isocyanatopropyltriethoxysilane, 3-isothiocyanonatopropyltriethoxysilane, 2-(4-chlorosulfonylphenyl) ethyltrimethoxysilane, 3-bromopropyltrimethoxysilane, methacryloxymethyltrimethylsilane; and the like. Polymer coating of glass-like and plastic surfaces may be achieved with polyvinyl alcohol, polyethyleneimine, polyacrolein, polyacrylic acid, etc. Direct chemical modification of plastic surfaces includes graft polymerization; halomethylation; plasma deposition of amines, alcohols, and carboxylic acids; nitration followed by reduction; and oxidation.

Coupler 1 (X): is an optional component, which is capable of attaching the cleavable linkage CL to Q, either indirectly or directly. Where X provides indirect coupling, it may be an affinity reagent comprising two binding partners. Examples of suitable binding partners include biotin/avidin or streptavidin; antibody/hapten; receptor/ligand pairs, as well as chemical affinity systems.

Where X provides direct coupling, the linkage may be a homo- or heterobifunctional linker having a group at one end capable of forming a stable linkage to Q, and a group at the opposite end capable of forming a stable linkage to the cleavable linker CL. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-y-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio)propionic acid N-hydroxysuccinimide ester (SPDP) or 4-(N-maleimidomethyl)-cyclohexane-1-carboxylic acid N-hydroxysuccinimide ester (SMCC).

Chemical groups that find use as couplings of Q to X include amide (amine plus carboxylic acid), ester (alcohol plus carboxylic acid), thioether (haloalkane plus sulfhydryl; maleimide plus sulfhydryl), Schiffs base (amine plus aldehyde), urea (amine plus isocyanate), thiourea (amine plus isothiocyanate), sulfonamide (amine plus sulfonyl chloride), and the like, as known in the art.

The species X is chosen to have properties that provide for stable attachment of CL to Q, as well as to provide good presentation of the capture system to the target species and to provide good accessibility of CL to the cleavage agent during the cleavage process. X comprises alkyl spacers, which may be linear or branched, usually linear, and may include one or more unsaturated bonds; having from one to about 12 carbon atoms. X also comprises spacers of this type having heteroatoms or functional groups present, including amines, ethers, phosphodiesters, and the like. Specific structures of interest include: (CH₂CH₂O)ₙ where n is from 1 to about 12; (CH₂CH₂NH)ₙ, where n is from 1 to about 12; [(CH₂)ₙ(C═O)NH(CH₂)ₘ]_{z}, where n and m are from 1 to about 6, and z is from 1 to about 10; [(CH₂)ₙOPO₃(CH₂)ₘ]_{z} where n and m are from 1 to about 6, and z is from 1 to about 10.

Cleavable linkage (CL): the cleavable linkage is a chemical linker that is not normally present on the targeted particles, and which is cleaved by an agent, which may be biological, e.g. enzymatic, chemical or physical, e.g. temperature, ionicity, light, pH, etc. One or more specific recognition sites may be present in CL; preferably, multiple sites having the same specificity are present in a single linker. Optionally, where two or more zones are present in a capture capillary, different cleavable linkages are present in each zone, where the specific recognition sequences are not cross-reactive with the other's cleavage agents.

Linkages of interest include DNA-DNA oligonucleotide hybrids; DNA-RNA oligonucleotide hybrids; RNA-RNA oligonucleotide hybrids; oligosaccharides; polypeptides; DNA-PNA hybrid (where PNA is a peptide nucleic acid); DNA-DNA hybrids plus minor groove binding polyamide; DNA-DNA hybrids plus major groove binding triple strand-forming oligonucleotide, etc.

Of particular interest is the use of oligonucleotides susceptible to cleavage with a specific nuclease, which may include recognition sequences for restriction endonucleases, as are well-known in the art. For example, the restriction enzyme EcoRI cleaves within the hexamer recognition sequence GAATTC, HindIII which cleaves within the hexamer recognition site AAGCTT, etc. DNA-RNA hybrids, which may be cleaved with, for example RNAse H, single stranded DNA or RNA, which is cleaved by S1 or micrococcal nuclease, etc. As described above, different zones in a capture capillary may comprise different recognition sequences allowing selective release of particular particle populations.

Alternative cleavable linkers of interest include oligosaccharides, for example dextran having an α(1 [symbol] 6) glycosidic linkage; cellulose having a β(1 [symbol] 4) glycosidic linkage; amylose; pectin; chitin, etc. Polypeptides of interest as cleavable linkers include those having an infrequently occurring sequence, or a sequence that is not normally present on an accessible surface of the particle. For example, proteases specific for sequences present in clotting factors may used, including factor Xa and thrombin.

As previously described, the binding system:

X′-CL-Y-L

may form a binding/specificity cassette that is used during manufacture of the capture capillaries. The coupler X comprising an affinity system is effectively divided into two binding pair members, X′ and X″. X′ is one member of a binding pair and X″ is the cognate binding pair member. The cassettes may be synthesized separately from the capillary system:

S-Q-X″

to provide greater flexibility and combinatorial diversity in the selective agents, i.e. CL and L.

In order to release particles that are bound to the selective ligand and thereby through the cleavable ligand to the capillary surface, a cleavage reagent is added that specifically cleaves the linker CL, while leaving substantially intact the binding moieties present on the target particle. Preferred cleavage reagents are enzymes that are reactive under physiological conditions, so as to minimize the effect on the target. After the release reaction, the viability of the cell, and/or particle is maintained, as is its ability to bind additional affinity reagents to particle surface markers, respond to agents through particle surface receptors, etc.

In one embodiment of the invention, the cleavage reagent is present in an encapsulated form bound to the capillary surface, and is released by pressure, or other means, preferably mechanical means.

Coupler 2 (Y): attaches the cleavable linkage CL to the selective binding ligand L. In general, the attributes described for X above apply equally to Y, that is, the species Y is chosen to have properties that provide for stable attachment of CL to L, as well as to provide good presentation of the capture system to the target species and to provide good accessibility of CL to the cleavage agent during the cleavage process. The required attachment can be achieved through biological affinity systems, which may include biotin/avidin or streptavidin; antibody/hapten; receptor/ligand; through chemical affinity (small molecule systems); or by direct covalent bonding. The particulars of attaching Y to CL and to L are the same as for attaching X to Q and CL.

Selective ligand (L): The term “specific binding member” as used herein refers to a member of a specific binding pair, i.e. two molecules, usually two different molecules, where one of the molecules through chemical or physical means specifically binds to the other molecule. The complementary members of a specific binding pair are sometimes referred to as a ligand and receptor. In addition to the frequently used antigen and antibody specific binding pairs, and peptide-MHC-antigen complexes and T cell receptor pairs, alternative specific binding pairs of interest include biotin and avidin or streptavidin; carbohydrates and lectins; complementary nucleotide sequences (including nucleic acid sequences used as probes and capture agents in DNA hybridization assays); peptide ligands and receptor; effector and receptor molecules; hormones and hormone binding protein; enzyme cofactors and enzymes; enzyme inhibitors and enzymes; and the like. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present.

Immunological specific binding pairs include antigens and antigen specific antibodies; and T cell antigen receptors, and their cognate MHC-peptide conjugates. Suitable antigens may be haptens, proteins, peptides, carbohydrates, etc. Recombinant DNA methods or peptide synthesis may be used to produce chimeric, truncated, or single chain analogs of either member of the binding pair, where chimeric proteins may provide mixture(s) or fragment(s) thereof, or a mixture of an antibody and other specific binding members. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

In a preferred embodiment, the selective ligand is an antibody or binding fragment derived therefrom. The antibody is directly or indirectly bound to Y. Where the antibody is present in the binding/selectivity cassette, it may be pre-bound to the targeted particles, or pre-bound to the capillary surface.

Antibodies of particular interest include those that recognize stem cells. For example, human hematopoietic stem cells may be positively selected using antibodies specific for CD34, thy-1, SCAH-1 and SCAH-2; or negatively selected using lineage specific markers which may include glycophorin A, CD3, CD24, CD16, CD14, CD38, CD45RA, CD36, CD2, CD19, CD56, CD66a, and CD66b; T cell specific markers, tumor specific markers, etc. Markers useful for the separation of mesodermal stem cells include FcγRII, FcγRIII, Thy-1, CD44, VLA-4α, LFA-1β, HSA, ICAM-1, CD45, Aa4.1, Sca-1, etc. Neural crest stem cells may be positively selected with antibodies specific for low-affinity nerve growth factor receptor (LNGFR), and negatively selected for the markers sulfatide, glial fibrillary acidic protein (GFAP), myelin protein Pₒ, peripherin and neurofilament. Human mesenchymal stem cells may be positively separated using the markers SH2, SH3 and SH4.

A number of other selective ligands are of interest, for example markers found on viruses, protozoan parasites, bacteria and other pathogens, and tumor specific antigens. For example, particular antigen-specific T cell subsets may be isolated wherein L is the cognate MHC-peptide for a particular range of T cell receptor αβ or T cell receptor γδ molecules.

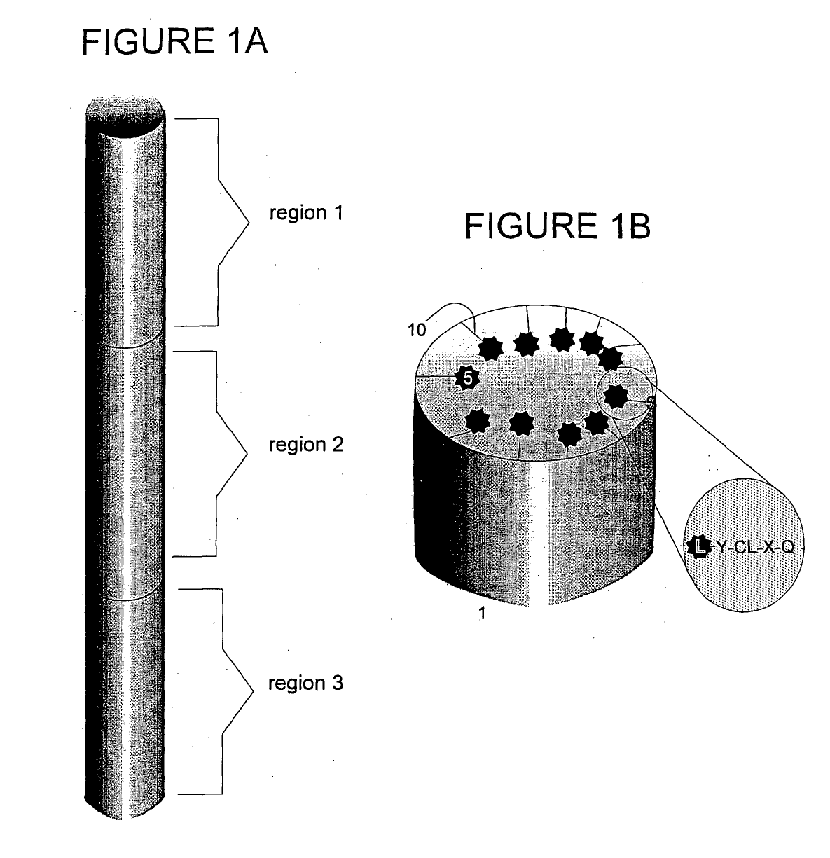

Capture capillaries: as used herein, is intended to encompass a capillary tubing coated on the luminal surface with a capture system as described above. The capillaries may be referred to in terms of an entrance end, an exit end, and a luminal surface positioned intermediate the entrance and exit ends and internal to the tubing. In operation, the sample fluid is provided to the entrance end of the capillary and is moved through the column by gravity flow, under pressure or by suction. As the fluid passes through the column, the capture system separates the target particle from the fluid composition. Typically the capillaries will have a round cross-sectional geometry, but other geometries, e.g. oval, may also be used. The basic capture capillary I is shown in FIG. 1B, having an internal surface with a capture system 10. In one embodiment of the invention, as shown in FIG. 1B, the capture capillary 1 is divided into multiple zones.

The zones may divide a unitary capillary, or each zone may be present on an individual capillary, which are then joined end-to-end in any convenient fashion. Each zone comprises a different selectivity reagent, either differing in the cleavable ligand, in the selectivity ligand, or in both. Where multiple zones are present, the zones may provide for either positive or negative selection. For example, in the separation of a rare species from a population, it may be desirable to have a first zone of a positive selection marker(s) and a second zone of a negative selection marker(s). Alternatively, the first and second zones may comprise different positive selection markers.

Where a zone comprises a negative selection marker, it is not necessary to include a capture system with a cleavable marker, because the targeted particles are not bound by the negative selection markers. Negative selection zones may comprise a separation system of the type:

S-Q-(X)ₙ-L

where the components are as previously described.

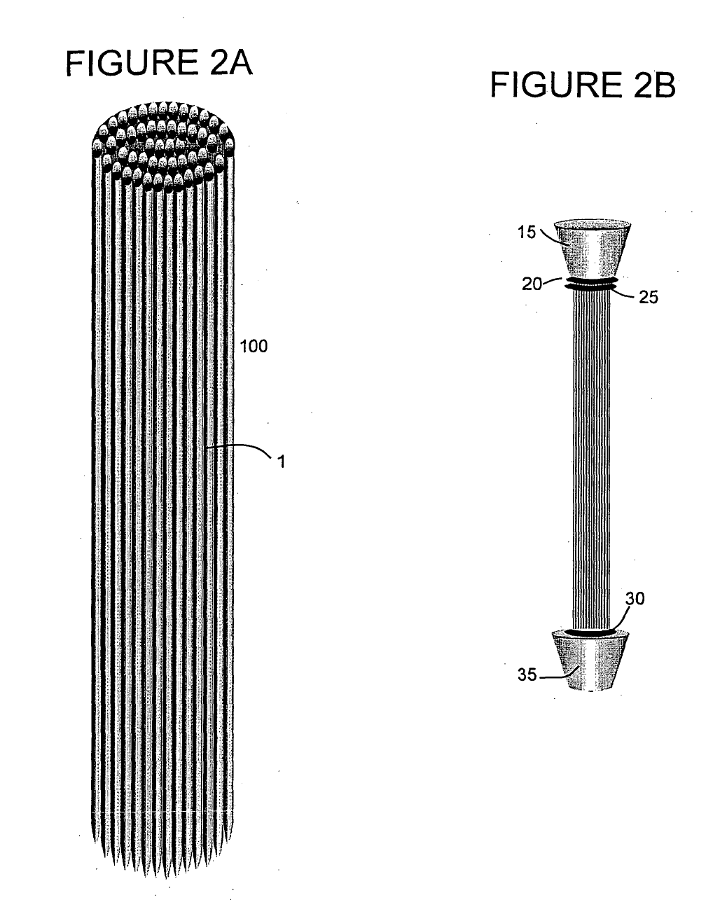

Capture device: As used herein, the term capture device is intended to refer to an array of capture capillaries as described above, and further comprising such filters, baffles, collection vessels etc, as required for performing a separation. A typical array 100 is shown in FIG. 2A. An array may comprise from about 10 to about. 10⁵ capillaries, more usually from about 10² to about 10⁴ capillaries. The number will be determined primarily on the number of particles and sample volume to be processed. The array may be arranged in an cross-sectional geometry convenient for the operator. Typically the “dead space” between capillaries will be minimized. In some embodiments, the dead-space may filled at the entrance to the capture device, to facilitate transfer of the sample into the capillaries.

As shown in FIG. 2B, at the inlet and outlet of the device, the array is coupled to a feeding mechanism 25 ensuring optimal homogeneous distribution and flow through the tubing. Optionally, a filter 20 may also be provided to prevent clumps of particles from entering the capillaries. A sample to be separated 15 is fed into the feeding mechanism. At the outlet of the device, a feeding mechanism 30 is provided to couple the device to a collection vessel 35 for the desired particle population.

Where the particles are a cell population for culture or for clinical use, the materials selected for the construction of the device will also be compatible with sterilization procedures. The sample 15 and collection vessel 35 may be closed vessels providing a sterile environment.

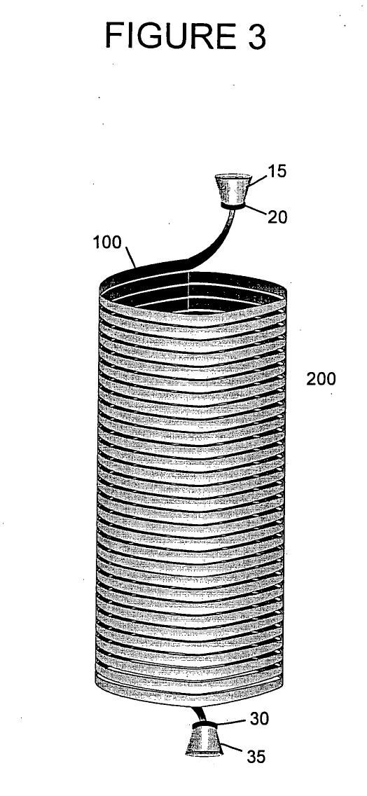

In one embodiment of the invention, the capillary array 100 is wrapped in a helical configuration, as shown in FIG. 3. The configuration provides a more convenient shape for longer tubing sets.

Capture instrument: In one embodiment of the present invention, an fluid control system is provided for use with the capture device, as described above. The fluid control system includes a column sensor for monitoring fluid flowing out of the device and into the collection vessel. The fluid control system may also include a device valve responsive to a valve control signal, for selectively enabling the fluid coming out of the device to flow into the collection vessel. A data processing means is provided for controlling the operation of the fluid control system. The data processor is responsive to the device signal for providing the valve control signal to optimize the concentrations of the target particles being collected.

The fluid control system may further include a pressure sensor coupled to the device for determining the pressure of the fluid in the device. The pressure sensor may include a connector for coupling a pressure signal to the data processor. A pump may be provided that is responsive to a pump control signal for controlling the speed and direction of fluid flow in the fluid tubing. The data processor is responsive to the pressure signal for providing the pump control signal to increase and decease the pressure of the fluid in the column.

The capture instrument may further include an agitation assembly for agitating the contents of the capture device to assist in the release of particles retained in the capillaries. The agitation assembly may be responsive to a drive signal for varying the amount of agitation of the contents of the device to vary the rate at which the target cells are released

### Separation of Particles from a Complex Mixture

The separation system of the present invention can be used to isolate any desired target substance. Of particular interest is the separation of a specific component from a complex mixture. The separation system of the present invention has great versatility, in that almost any target substance may be separated once a specific binding member is available. The target substance or analyte may be any member of a specific binding pair, or a substance associated with a member of a specific binding pair. As an example, a cell surface antigen-antibody binding pair may be used to isolate the antigen itself, cells that express the antigen, a particular organelle involved in processing of the antigen, etc. The devices and methods of the present invention are also advantageously applied to diagnostic techniques involving the binding of a receptor and ligand, such as immunoassays, and the like.

For brevity, the separation system will mainly be described in terms of its ability to specifically select and separate a defined population of cells (target cells) from a mixed cell population, such as peripheral blood, bone marrow, blood from the umbilical cord or placenta, fetal blood or a leukopheresis product. It will also be appreciated that some tissues may be disrupted into a single cell or monodisperse suspension to allow isolation of a particular cell subset, such as the separation of tumor infiltrating lymphocytes from a tumor mass, the separation of islet cells from pancreatic tissue, etc. For example, different cell types may be labeled with a specific antibody to allow cell purging and/or cell enrichment. The target cell population is generally identified by a specific binding member, as described above, which selectively binds to a cell surface antigen present on the target cells. It should be understood, however, that the subject apparatus and method is not limited to such uses.

Various methods and devices exist for pre-separating component parts of a sample fluid to obtain target particles. These methods include filters, centrifuges, chromatographs, and other well-known fluid separation methods. These methods include gross separation using columns, centrifuges, filters, separation by killing of unwanted cells, separation with fluorescence activated cell sorters, separation by directly or indirectly binding cells to a ligand immobilized on a physical support, such as panning techniques, separation by column immunoadsorption, and separation using magnetic immunobeads.

After a pre-selection process, the sample of cells may be pre-bound to one or more positive selectivity cassettes of the type X′-CL-Y-L. Methods of binding such reagents to cells are well known in the art. A sufficient quantity of the selection cassette will be added such that the reagent is not a limiting factor in the binding reaction. The binding will take from about 5 to 30 minutes. The excess selectivity cassette is the washed from the cells. Where the population is pre-bound in this manner, the capture device will comprise the capture system S-Q-X″, where X′ and X″ are cognate members of a binding pair.

Alternatively, the sample is not pre-bound, but is directly applied to a capture device, where the capture device comprises a complete capture system, S-Q-(X)ₙ—CL-(Y)ₘ-L.

The cell population is applied to the capture device in a fluid, under flow conditions that minimize shear forces, but which maximize the contact of the cells with the capture surfaces. The fluid may be any acceptable buffer system. Where the targeted particles are cells, the fluid will be chosen to maintain the physiological integrity of the cells, preferably maximizing cell viability.

Once the targeted cells are bound to the capture device and the undesirable cells washed away, the cleavage reagent is added to the capture device. The reagent is typically flowed through the device in a suitable medium. The concentration of cleavage reagent will be sufficient to cleave substantially all of the cleavage linker, usually at least about 90%, more usually at least about 95%, and preferably at least about 99%. The conditions for release may be empirically optimized in terms of temperature, pH, presence of metal cofactors, reducing agents, etc. by varying such conditions and determining the quantitative effect on cells release. The release will usually be complete in at least about 15 minutes, more usually at least about 10 minutes, and will usually not be longer than about 30 minutes.

On completion of the cleavage step, the cells are released form the capillary wall. The passage of cells is controlled through the zones. For example, the flow of cells may be allowed to proceed to a first zone, at which point the flow is stopped to allow binding to the selective linker, permit cleavage to proceed, etc. Once the selection process is completed, the flow is resumed to a second zone, where further selection takes place.

Where the capture device comprises multiple zones, the cells will be contacted with the lower zones after release from the initial zone. The contact, binding and washing steps are performed as described above for the first zone. Preferably a second positive selection marker will comprise a different cleavage specificity, and the bound cells will be released after cleavage of the second linker. Where a second or later zone comprises a negative selection marker, the targeted cells will flow through, and there is no requirement for a cleavage reagent.

The medium in which the cells are released will be any medium that maintains the viability of the cells and allows activity of the release agent. Suitable media include phosphate buffered saline containing from 0.1 to 0.5% BSA, Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The release method is useful during purification of specific cell types from complex mixtures, where the procedure has multiple separation steps. The procedure may use a variety of combinations of enrichment and depletion steps, generally starting with an enrichment. In order to achieve a high degree of purification for a single marker, two sequential enrichment steps may be performed, using antibodies specific for two different epitopes of the marker, for example, two different anti-CD34 antibodies. Two sequential positive selections may be performed for different markers, for example, combining separations for two or more of CD34, thy-1, CD71, transferrin receptor, HB-F fetal cell selection, a cocktail of hematopoietic lineage markers, such as a combination of CD4/CD8/CD19, cytokine receptors, CD45RA/RO, etc. It is often desirable to first perform an enrichment when sorting a minor cell population from a complex mixture, in order to reduce the number of cells being manipulated. For example, an enrichment for CD34 positive cells during selection for hematopoietic progenitor cells, or an enrichment for CD71 positive cells during selection for fetal cells in maternal blood, are useful first steps. This enrichment is then followed by a depletion step, and optionally, another positive selection. The number of selection steps can be extended as necessary.

In many cases analysis will be performed on aliquots of the original and separated populations to follow the separation procedure. It may be useful to label the antibodies with a fluorochrome, e.g. phycoerythrin, FITC, rhodamine, Texas red, allophycocyanin, etc. The fluorochrome label may be used to monitor microscopically or by flow cytometry the cell composition after the separation steps. Fluorescent labeling may conveniently utilize the same indirect coupling system as the particles.

In order to address the needs of research and clinical laboratories, a kit may be provided having the reagents and apparatus necessary to perform the subject invention. Such a kit will contain the capture device having the appropriate coating, optionally in combination with a selectivity cassette. The cleavage reagent for the various cleavable linkers may also be included. Other components provided may be pre-separation reagents, instrument components, buffers for enzyme digestion, etc. While single arrays may be used, it is anticipated that multiple arrays may be run simultaneously, and an apparatus for automated or manual procedures may optionally be provided for such a purpose.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications mentioned herein are incorporated herein by reference for the purpose of describing and disclosing, for example, the compounds and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### EXPERIMENTAL

### Example 1

### Isolation of Human Hematopoietic Stem Cells and Breast Cancer Specific T Cells

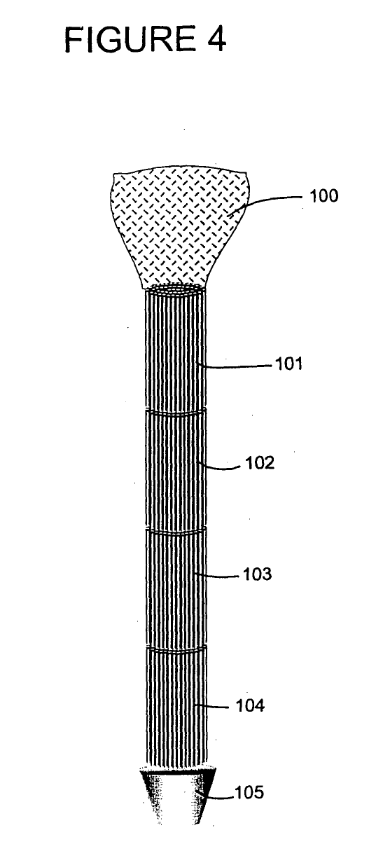

Human hematopoietic stem cells (HSC) and breast cancer specific T cells are sequentially isolated from a patient sample in a single process. An illustration of the separation apparatus, including capillary zones, is provided in FIG. 4.

In order to readily isolate HSC, the donor is treated with a mobilizing agent prior to collection. PBMC are obtained from leukocyte-rich buffy coats by centrifugation over Ficoll-Paque (Pharmacia, Uppsala, Sweden). After centrifugation, interphase cells are collected, resuspended in buffer and sedimented at 300×g and then once again resuspended in buffer and centrifuged at 200×g to remove platelets.

A pre-separation is performed in negative selection bag 100. The bag contains a fine mesh for high capacity negative selection. Monoclonal antibodies are bound to the mesh. Antibodies used for selection react specifically with B cell, red blood cell, macrophage, monocyte, breast epithelium, platelet, or other lineage markers that are not present on T cells. The cells are incubated in the bag for at least one hour.

The unbound cells are then moved to zone 1 (101) through controlled flow. Zone 1 comprises a capillary array where the cleavable linker is a double stranded DNA oligomer comprising multiple repeats of the recognition sequence for EcoRI. The selective ligand L is a major histocompatibility complex protein, of HLA-A type with the peptide antigen her2/neu. This zone binds the T cells that are specific for breast cancer cells bearing the her2/neu surface antigen. The cells are incubated in the zone 1 section for approximately 30 minutes.

The unbound cells are then moved to zone 2 (102) through controlled flow. Zone 2 comprises a capillary array where the cleavable linker is a double stranded DNA oligomer comprising multiple repeats of the recognition sequence for Hpa II. The selective ligand L is a monoclonal antibody specific for CD34. This zone binds the hematopoietic progenitor cells bearing the CD34 surface antigen. The cells are incubated in the zone 2 section for approximately 30 minutes.

After incubation, the cells are washed thoroughly to release unbound cells throughout the capillary array. The releasing agent for Zone 2 is then applied, in order to release the lineage negative, CD34 positive cells. The cells are released by incubation of the cells in zone 2 with Hpa II restriction enzyme for a period of about one hour.

The released cells are washed free of zone 2, and moved to zone 3 (103) through controlled flow. Zone 3 comprises a capillary array where the cleavable linker is a double stranded DNA oligomer comprising multiple repeats of the recognition sequence for Msc I. The selective ligand L is a monoclonal antibody specific for Thy-1. This zone binds the hematopoietic progenitor cells bearing the Thy-1 surface antigen. The cells are incubated in the zone 3 section for approximately 30 minutes.

After incubation, the cells are washed thoroughly to release unbound cells throughout the capillary array. The releasing agent for Zone 3 is then applied, in order to release the lineage negative, CD34 positive, Thy-1 positive cells. The cells are released by incubation of the cells in zone 3 with Msc I restriction enzyme for a period of about one hour.

The released cells are washed free of zone 3, and moved to zone 4 (104) through controlled flow. Zone 4 is a negative selection array, having the same panel of lineage markers as were present in the high capacity negative selection bag. The cells are incubated in the zone 4 section for approximately 30 minutes. The unbound cells are washed through, and collected. This fraction comprises the hematopoietic stem cells, which are used for reconstitution of patient hematopoietic function, usually after ablative chemo- or radiotherapy.

After the HSC are collected, the T cells are released by incubation with the releasing agent for zone 1, which is EcoRI. The cells are incubated for a period of about one hour in the releasing agent, and are then flowed to zone 4. In zone 4 the cells undergo a second negative selection step by incubation for a period of approximately 30 minutes. The unbound cells are washed through and collected. This fraction represents the T cells recognizing breast cancer cells, and are used for immunotherapy.

For further analysis of the cells, in order to determine purity, they are assessed by flow cytometry. 1 μl (1 mg/ml) of propidium iodide is added to the sample and the cells are analyzed on a FACScan Flow Cytometer (Becton Dickinson). Debris and dead cells are excluded by gating.

### Example 2

### Amination of Fused-Silica Capillary Surface

Ten (10) fused-silica capillaries (250 μm i.d., 350 μm o.d., 1 meter length; Polymicro Technologies) are each pierced through a rubber septum (24/40 standard taper joint size; Aldrich Chemical) using a 16-gauge needle. The capillaries are adjusted to protrude about three (3) inches from the bottom of the septum. The septum with capillaries is then fitted to the top joint of a 24/40 straight vacuum adapter (ChemGlass). This assembly is then placed onto a one-liter 24/40 round-bottom flask, and a vacuum source is attached to the vacuum adapter. The free ends of the capillary are placed into a 100-ml graduated cylinder containing 100 ml of 0.1 M aqueous sodium hydroxide solution. This solution is drawn through the capillaries under vacuum. The capillaries are then washed with 100 ml of distilled, deionized water using the same procedure. Finally, the capillaries are dried by drawing dry nitrogen through them under vacuum for thirty (30) minutes.

100 ml of a 2.5% (v/v) solution of 2-aminopropyltriethoxysilane (Acros) in reagent-grade acetone is prepared in a 125 ml Erlenmeyer flask. The flask is positioned about 30 cm above the capillary-septum-adapter assembly. The free ends of the dried capillaries are placed in the solution, and flow is started by a short application of vacuum to the system. Once flow is observed in all capillaries, the vacuum is removed and the solution perfused through the capillaries under gravity flow for thirty (30) minutes. Approximately 70 ml of solution flows through the capillaries during this time. The free ends of the capillaries are removed from the flask, and dry air is drawn through them for fifteen (15) minutes. The capillaries are removed from the septum and placed in an oven at 95° C. overnight to cure.

### Example 3

### Biotinylation of Aminated Fused-Silica Capillary Surface

The ten (10) aminated fused-silica capillaries from Example 2 are each pierced through a rubber septum (24/40 standard taper joint size) using a 16-gauge needle. The capillaries are adjusted to protrude about three (3) inches from the bottom of the septum. The septum with capillaries is then fitted to the top joint of a 24/40 straight vacuum adapter. This assembly is then placed onto a 500 ml 24/40 round-bottom flask, and a vacuum source is attached to the vacuum adapter.

A solution of 100 mg of Biotin-X-NHS (Molecular Probes) in 10 ml of 9:1 anhydrous N-methylpyrrolidinone:N,N-diisopropylethylamine (Aldrich Chemical) is prepared. This solution is added to a 15 ml conical plastic centrifuge tube which is placed about thirty (30) cm above the capillary-septum-adapter assembly. The free ends of the aminated capillaries are placed in the solution, and flow is started by a short application of vacuum to the system. Once flow is observed in all capillaries, the vacuum is removed and the solution perfused through the capillaries under gravity flow until all of the solution has flowed through the capillaries. The capillaries are then sequentially washed under vacuum with 10 ml of N-methylpyrrolidinone, 10 ml of denatured ethanol, and 10 ml of water. The free ends of the capillaries are removed from the tube, and dry air is drawn through them for fifteen (15) minutes. The biotinylated capillaries are removed from the septum and placed in a plastic bag at 4° C. until needed.

### Example 4

### Testing of Streptavidin Binding to Biotinylated Capillary Surface

A biotinylated capillary from Example 3 is attached to a 30 ml plastic syringe (Becton Dickinson) held in a syringe pump (KD Scientific, Model 100). The mating of the capillary to the Luer end of the syringe is accomplished by fitting a female Luer-to-male Fingertight fitting adapter (Upchurch Scientific), and attaching a female Fingertight fitting-to-Microtight fitting adapter (Upchurch Scientific) to it. The capillary is then joined via the Microtight fitting.

The capillary is washed with 10 ml of PBS buffer (0.1 M sodium phosphate, 0.15 M sodium chloride, pH 7.2) using the syringe pump to deliver buffer at 20 ml/hour. The 30 ml syringe is replaced with a 1 ml plastic syringe (Becton Dickinson) containing 1 ml of a 10 mg/ml solution of bovine serum albumin (BSA; Sigma) in PBS. The capillary is perfused with this solution using the syringe pump to deliver solution at 2 ml/hour. The BSA treatment is used to minimize the non-specific binding of biologicals (e.g., antibodies, nucleic acids, cells) to the capillary surface in subsequent manipulations.

An alkaline phosphatase substrate solution is made up using 15 ml of 2×DEA buffer (2 M diethanolamine, 2 mM MgCl₂, 0.2 mM ZnCl₂) and 15 ml of water in which one p-nitrophenyl phosphate (PNPP) tablet (Sigma) is dissolved.

Control Experiment.

The BSA-blocked capillary is washed with 10 ml of PBS using the syringe pump to deliver buffer at 20 ml/hour. A solution of 0.5 mg of alkaline phosphatase in 1.0 ml of PBS containing 1 mg/ml BSA is prepared and perfused through the capillary at 0.5 ml/hour. The capillary is washed twice with 30 ml aliquots of PBS at 30 ml/hour. Finally, 0.5 ml of the above PNPP solution is pumped through the capillary at 10 ml/hour. The solution exiting the free end of the capillary is a very pale yellow, indicating little non-specific alkaline phosphatase binding to the biotinylated capillary surface.

Binding Experiment:

The same capillary that was used in the control experiment is washed with 60 ml of PBS at 30 ml/hour. A solution of 1 mg of streptavidin-alkaline phosphatase conjugate (Sigma) in 1.0 ml of PBS is prepared and perfused through the capillary at 0.5 ml/hour. The capillary is washed twice with 30 ml aliquots of PBS at 30 ml/hour. Finally, 0.5 ml of the above PNPP solution is pumped through the capillary at 10 ml/hour. The solution exiting the free end of the capillary is a bright yellow, indicating significant alkaline phosphatase binding to the biotinylated capillary surface by virtue of the-specific streptavidin-biotin interaction.

### Example 5

### Preparation of Streptavidin-Coated Capillary

A biotinylated capillary from Example 3 is attached to a 30 ml plastic syringe (Becton Dickinson) held in a syringe pump (KD Scientific, Model 100). The mating of the capillary to the Luer end of the syringe is accomplished by fitting a female Luer-to-male Fingertight fitting adapter (Upchurch Scientific), and attaching a female Fingertight fitting-to-Microtight fitting adapter (Upchurch Scientific) to it. The capillary is then joined via the Microtight fitting.

The capillary is washed with 10 ml of PBS buffer (0.1 M sodium phosphate, 0.15 M sodium chloride, pH 7.2) using the syringe pump to deliver buffer at 20 ml/hour. The 30 ml syringe is replaced with a 1 ml plastic syringe (Becton Dickinson) containing 1 ml of a 10 mg/ml solution of bovine serum albumin (BSA; Sigma) in PBS. The capillary is perfused with this solution using the syringe pump to deliver solution at 2 ml/hour. The BSA treatment is used to minimize the non-specific binding of biologicals (e.g., antibodies, nucleic acids, cells) to the capillary surface in subsequent manipulations.

The 1 ml syringe is replaced with the 30 ml syringe, and the BSA-blocked capillary is washed with 30 ml of PBS using the syringe pump to deliver buffer at 30 ml/hour. The 30 ml syringe is replaced with the 1 ml syringe containing 1 ml of a solution of 2.5 mg/ml streptavidin (Sigma) and 1 mg/ml BSA in PBS. The capillary is perfused with this solution at a rate of 0.5 ml/hour. Finally, the 1 ml syringe is replaced with the 30 ml syringe and the capillary is washed with 30 ml of PBS at a rate of 20 ml/hour.

The streptavidin-coated capillary is stored in a plastic bag with a little water in it at 4° C.

### Example 6

### Preparation of Oligonucleotides

The following two complementary oligodeoxyribonucleotide sequences containing a single EcoRI restriction enzyme cleavage site (shown in bold letters) are used in this example:

| | | |
|---|---|---|
| EcoRI | 5′>AGT AAC ACG GTC ATC GAA TTC CAG CCT TAG AAG CTT<3′ | (SEQ ID NO:1) |
| EcoRIC | 3′>TCA TTG TGC CAG TAG CTT AAG GTC GGA ATC TTC GAA<5′ | (SEQ ID NO:2) |

Four 1 μmole syntheses of EcoRI are carried out using standard DNA synthesis techniques (PE Biosystems Model 394 automated DNA synthesizer with manufacturer's synthesis cycles; reagents from Glen Research). The sequences are prepared in Trityl On, Manual cleavage mode. The oligonucleotides are then 5′-end labeled on the DNA synthesizer with first a hexaethylene glycol spacer and then biotin, using commercial phosphoramidite reagents (Glen Research). The synthesis cycle is modified to provide a ten minute coupling time for these non-nucleotidic phosphoramidites. The final sequences are prepared in Trityl On, Auto cleavage mode, and deprotected overnight at 60° C. The crude tritylated oligonucleotides are then obtained by concentrating the cooled deprotection solution to about 0.5 ml in a SpeedVac (Savant Instruments).

The crude oligonucleotides are purified twice by reverse phase HPLC on an Inertsil C4 5 μm HPLC column, 4.6 mm×150 mm (MetaChem Technologies), using a 1050Ti HPLC system (Agilent/Hewlett Packard) connected to a Foxy II fraction collector (Isco). The purifications are run using a linear gradient of acetonitrile in 0.1 M triethylammonium acetate, pH 6.5. The flow rate is 1.0 ml/minute. The effluent is monitored at 280 nm. The initial purification is performed on 100 μl aliquots of the crude product solution. Following the first HPLC purification, the fractions containing the purified tritylated oligonucleotides are evaporated to dryness in a SpeedVac. The dried pellets are treated with 1 ml of 80:20 (v/v) acetic acid:water for 60 minutes at room temperature to remove the trityl groups, then evaporated to dryness in a SpeedVac. The dried oligonucleotides are dissolved in 0.5 ml of water, and again HPLC purified in 250 μl aliquots. Finally, the fractions containing the pure oligonucleotides are evaporated to dryness in a SpeedVac, dissolved in a total volume of 1 ml of water, and stored frozen. The concentration of the stock oligonucleotide solution is determined by diluting 5 μl of the stock into 1 ml of water, measuring the absorbance of this solution at 260 nm, and calculating the concentration using Beer's law (A=εbc with ε=36×10⁴ M⁻¹ cm⁻¹, b=1 cm, and a dilution factor of 200).

Four 1 μmole syntheses of EcoRIC are carried out using standard DNA synthesis techniques as described in the previous paragraph. Oligonucleotides are prepared in Trityl On, Manual cleavage mode. Two 1 μmole syntheses are then 5′-end labeled on the DNA synthesizer with first a triethylene glycol spacer and then 5-amino-3-oxo-1-pentanol, using commercial phosphoramidites (Glen Research). The other two 1 μmole syntheses are 5′-end labeled with fluorescein using a commercial phosphoramidite (Glen Research). The oligonucleotides are deprotected and purified as in the above paragraph, except that the fluorescein-labeled oligonucleotides do not require detritylation between HPLC runs, as they have no trityl group. The pure oligonucleotides are stored frozen. The concentration of the stock oligonucleotide solutions are determined by diluting 5 μl of the stock into 1 ml of water, measuring the absorbance of this solution at 260 nm, and calculating the concentration using Beer's law (A=εbc with ε=36×10⁴ M⁻¹ cm⁻¹, b=1 cm, and a dilution factor of 200).

### Example 7

### Testing of Biotinylated Oligonucleotide Binding to Streptavidin-coated Capillaries

A streptavidin-coated capillary from Example 5 is washed with 30 ml of PBS (0.1 M sodium phosphate, 0.15 M sodium chloride, pH 7.2) using the syringe pump to deliver buffer at 30 ml/hour.

A solution of 20 μl of stock oligonucleotide Biotin-EcoRI (˜5.8 nmoles) in 20 ml of PBS is prepared. Ten ml of this solution are perfused through the above capillary at 2 ml/hr. One ml fractions are collected in 1.5 ml microcentrifuge tubes. The fractions are assayed by measuring the UV absorbance at 260 nm. The amount of biotinylated oligonucleotide removed from the solution as it passes through the capillary is calculated using Beer's law (see Example 6). Most of the oligonucleotide is removed in fraction 1. The total amount of oligonucleotide bound is calculated to be ˜20 pmoles per meter of capillary.

### Example 8

### Testing of Complementary Oligonucleotide Binding to Oligonucleotide-coated Capillaries

The capillary coated with biotinylated oligonucleotide from Example 6 is washed with 30 ml of PBS (0.1 M sodium phosphate, 0.15 M sodium chloride, pH 7.2) using the syringe pump to deliver buffer at 30 ml/hour. The capillary is then removed from the syringe pump and cut into two equal pieces. One half is stored in a plastic bag with a little water in it at 4° C. The other half is re-affixed to the syringe pump.

A solution of 1 μl of stock oligonucleotide Fluorescein-EcoRIC (˜375 pmoles) in 40 ml of PBS is prepared. Ten ml of this solution are perfused through the above capillary at 4 ml/hr. One ml fractions are collected in 1.5 ml microcentrifuge tubes. The fractions are assayed by measuring the UV absorbance at 260 nm. The amount of complementary oligonucleotide removed from the solution as it passes through the capillary is calculated using Beer's law (see Example 6). Most of the oligonucleotide is removed in fractions 1-3. The total amount of complementary oligonucleotide hybridized is calculated to be ˜50 pmoles per meter of capillary.

### Example 9

### Testing of Double-stranded Oligonucleotide Binding to Streptavidin-coated Capillaries

A streptavidin-coated capillary from Example 5 is washed with 30 ml of PBS (0.1 M sodium phosphate, 0.15 M sodium chloride, pH 7.2) using the syringe pump to deliver buffer at 30 ml/hour.

A solution of 2.0 ul of stock oligonucleotide Biotin-EcoRI (˜580 pmoles) and 1.6 ul of stock oligonucleotide Fluorescein-EcoRIC (˜600 pmoles) in 20 ml of PBS is prepared and allowed to sit for thirty (30) minutes at room temperature. Six ml of this solution are perfused through the streptavidin-coated capillary at 4 ml/hour. One ml fractions are collected in 1.5 ml microcentrifuge tubes. The fractions are assayed by measuring the UV absorbance at 260 nm. The amount of double-stranded oligonucleotide removed from the solution as it passes through the capillary is calculated using Beer's law (see Example 5). Most of the oligonucleotide is removed in fractions 1-3. The total amount of double-stranded oligonucleotide bound is calculated to be ˜60 pmoles per meter of capillary.

### Example 10

Preparation of Oligonucleotide-Conjugated Antibody

Oligonucleotide Modification: Four hundred microliters of a stock solution of amino-EcoRIC (˜380 nmoles) is mixed with 400 μl of 1 M MOPS(Na), pH 7.3, buffer. A solution of 2.0 mg (FW 312.4, ˜6.4 μmoles, ˜16.6-fold excess) of N-succinimidyl 3-(2-pyridyldithio)propionate (SPDP; Pierce) in 200 μl of dry methyl sulfoxide (Aldrich) is added. The clear solution is shaken at room temperature for 1 hour, and then allowed to sit an additional 2.5 hours at 4° C. The reaction mixture is purified on a NAP-25 gel filtration column (Amersham Pharmacia) equilibrated with and eluting with PBSE (0.1 M sodium phosphate, 0.15 M sodium chloride, 1 mM EDTA, pH 7.4). Eight 1 ml fractions are collected in 1.5 ml microcentrifuge tubes and assayed for DNA by UV spectroscopy. Fractions 3-6 containing DNA are pooled.

The SPDP-modified oligonucleotide is analyzed by reverse phase HPLC on an Inertsil C4 5 μm, 4.6 mm×150 mm column (MetaChem Technologies) using a linear gradient of acetonitrile in 0.1 M triethylammonium acetate, pH 6.5. Approximately 80% conversion of the amino-oligonucleotide to the pyridyldithiopropionyl-oligonucleotide is observed, as estimated from relative peak areas for the two species appearing in the chromatogram.

The concentration of the stock pyridyldithiopropionyl-oligonucleotide is determined by diluting 5 μl of the stock into 1 ml of water, measuring the absorbance of this solution at 260 nm, and calculating the concentration using Beer's law (A=εbc with ε=36×10⁴ M⁻¹ cm⁻¹, b=1 cm, and a dilution factor of 200). A value of 60.6 μM is obtained for this preparation.

The extent of SPDP-modification is determined as follows. A 100 μl aliquot of the stock modified oligonucleotide solution (˜6.1 nmoles) is with 900 μl of PBSE and this solution is introduced into a UV spectrometer cuvette. Ten microliters of a 10 mM solution of tris-carboxyethylphosphine hydrochloride (TCEP) (100 nmoles; Pierce) in water is added, and the absorbance of the reaction mixture at 343 nm is measured over fifteen minutes. The absorbance rapidly increases over the first few minutes, indicating the reduction of the pyridyldisulfide by TCEP with the concomitant production of the chromophore pyridine-2-thione and thiolated-oligonucleotide, and then levels off. The concentration of pyridine-2-thione, and hence the concentration of SPDP-modified oligonucleotide, is calculated from the maximum value of the absorbance at 343 nm using Beer's law (ε₃₄₃=8.08×10³), and is 52.0 μM for this preparation. The percent modification of oligonucleotide is thus 86%, in good agreement with the chromatography result.

Antibody Modification: One ml of a 0.5 mg/ml solution of antibody M1/70 (˜3.3 nmoles; provided by Stanford Medical School) in PBS is mixed with 100 μl of 1 M MOPS(Na), pH 7.3, buffer. A solution of 0.1 mg of sulfosuccinimidyl 4-(N-maleimidomethyl)cyclohexane-1-carboxylate (Sulfo-SMCC, ˜228 nmoles, ˜69-fold molar excess) in 25 μl of dry methyl sulfoxide (Aldrich) is added. The clear solution is shaken at room temperature for 2 hours. The reaction mixture is purified on a NAP-gel filtration column (Amersham Pharmacia) equilibrated with and eluting with PBSE (0.1 M sodium phosphate, 0.15 M sodium chloride, 1 mM EDTA, pH 7.4). Eight 1 ml fractions are collected in 1.5 ml microcentrifuge tubes and assayed for protein by UV spectroscopy. Fractions 4 and 5 containing IgG are pooled. It is assumed that essentially all IgG is recovered in this process.

Preparation of DNA-Antibody Conjugate: One milliliter of the above SPDP-modified oligonucleotide solution (˜52 nmoles of modified oligonucleotide) is added to a quartz cuvette, and the cuvette is placed in the UV spectrometer. A solution of 20 μg of TCEP in 10 μl of water is added, and the absorbance at 343 nm is monitored as above. The rate of the reaction slows to near zero by 20 minutes. After 1 hour, the solution containing the thiolated-oligonucleotide is removed from the cuvette, and 100 μl (˜5.2 nmoles) is added to the solution of SMCC-modified IgG described above. The reaction is allowed to proceed overnight at 4° C.

The oligonucleotide-IgG conjugate is purified using Ultrafree-MC centrifugal filter units (Millipore), made from regenerated cellulose and having a nominal molecular weight retention above 30,000 daltons. These ultrafiltration units will thus retain the protein-DNA conjugate, while allowing any unreacted oligonucleotides to pass through the membrane to waste. Two units are first blocked with BSA to reduce loss of the conjugate through non-specific binding to the membrane. Four hundred microliters of 10 mg/ml of BSA in PBS are pipetted into each unit. The units are placed in a refrigerated centrifuge (model GS-15R; Beckman Instruments) at 4° C., and spun at 3000×g for 30 minutes. The sample cups are then rinsed with water and transferred to fresh microcentrifuge tubes. Four hundred microliters of the crude oligonucleotide-IgG conjugate solution are added to each unit. The units are placed in the refrigerated centrifuge at 4° C., and spun at 3000×g for 30 minutes. The flow-through is discarded, and the remainder of the conjugation reaction is divided equally between the two units. The retained conjugate solutions are diluted to 400 μl with PBS, and the units are again spun at 3000×g for 30 minutes at 4° C. The flow-through is again discarded, the retained conjugate solutions diluted to 400 μl with PBS, and the units are spun at 3000×g for 30 minutes at 4° C. This process is repeated four more times. Finally, the retained conjugate solutions are diluted to 400 μl with PBS, and combined in a 1.5 ml microcentrifuge tube. The concentration of conjugate is determined by measuring the absorbance of this solution at 260 nm, and calculating the concentration using Beer's law (A=εbc with ε=36×10⁴ M⁻¹ cm⁻¹, b=1 cm). The concentration for this preparation is 42.2 μM.

### Example 11

### Enrichment for Mac-1 Positive Cells

To enrich for cells expressing high levels of Mac-1 on the surface, a preparation is run on the apparatus as described above.

One meter of capillary with 250 μm inside diameter was connected to a circulation pump. The capillary tube itself was coated with biotin as described in Example 3. A solution of streptavidin at 5 mg/ml in PBS+0.5% BSA was circulated through the tubing for 30 min. The capillary was then washed with 3 ml of PBS+BSA for 15 min.

As described in Example 7, the biotinylated oligonucleotide (SEQ ID NO:1) at a concentration of 3 μM in PBS+BSA was circulated through the streptavidin coated capillary. The capillary is washed for 15 min with PBS+BSA.

To attach the Mac-1 antibody to the capillary (as described in Example 10), antibody conjugated to an oligonucleotide (Oligo2 (SEQ ID NO:2)-Mac-1_Antibody) was heated to 37° C. for 10 min. A 2 μM solution of antibody was run through the capillary for 30 min at 37° C., then washed for 0.15 minutes.

A suspension of freshly isolated splenocytes at a concentration of 3×106 in PBS+BSA were circulated through the capillary. The tubing was then washed for 15 minutes. The pump (but not the capillary with cells) was washed extensively to clear out any remaining unbound cells, and a second antibody added as a marker (Goat-Anti-Rat IgG) by running through the capillary for 20 min., then washing for 15 minutes.

To elute the bound cells, 200 units of EcoR1 endonuclease in 1×ReACT 3 buffer plus BSA was recirculated through the capillary for 35 minutes at 37° C. The cells were eluted with an additional 300 μl of PBS+BSA, and collected for FACS analysis.

What is claimed is:

1. A capture capillary for separation of biological particles from a complex mixture, the capillary comprising:
a capture system on the luminal surface having the formula
S-Q-(X)ₙ-CL-(Y)ₘ-L
wherein S is said luminal surface of the capillary tubing; Q is a chemical linkage between the surface and X; X is an affinity reagent; n is 1 or 0; CL is a cleavable linkage; Y is a linker or affinity reagent; m is 1 or 0; and L is a ligand capable of specifically binding a moiety present on a desired particle subset.

2. The capture capillary of claim 1, wherein n is 1; and X is an affinity reagent comprising cognate binding pair members X′ and X″.

3. The capture capillary of claim 1, wherein said capillary is divided linearly into multiple zones, wherein each of said zones comprises a unique L ligand.

4. The capture capillary of claim 1, wherein said cleavable ligand CL comprises an oligonucleotide.

5. The capture capillary of claim 4, wherein said oligonucleotide is double stranded DNA comprising one or more recognition sequences for a restriction enzyme.

6. The capture capillary of claim 4, wherein said olignucleotide is a double stranded RNA:DNA hybrid.

7. The capture capillary of claim 1, wherein said ligand L recognizes a cell surface antigen.

8. The capture capillary of claim 7, wherein said ligand L is an antibody.

9. The capture capillary of claim 1, wherein (we should include some claims for density of capture systems).

10. A-capture device, comprising:
an array of from 10 to 10⁵ capture capillaries the capillary comprising:
a capture system on the luminal surface having the formula
S-Q-(X)ₙ-CL-(Y)ₘ-L
wherein S is said luminal surface of the capillary tubing; Q is a chemical linkage between the surface and X; X is an affinity reagent; n is 1 or 0; CL is a cleavable linkage; Y is a linker or affinity reagent; m is 1 or 0; and L is a ligand capable of specifically binding a moiety present on a desired particle subset.

11. The capture device of claim 10, wherein n is 1; and X is an affinity reagent comprising cognate binding pair members X′ and X″.

12. The capture device of claim 10, wherein said capillary is divided linearly into multiple zones, wherein each of said zones comprises a unique L ligand.

13. The capture device of claim 10, wherein said cleavable ligand CL comprises an oligonucleotide.

14. The capture device of claim 10, wherein said ligand L recognizes a cell surface antigen.

15. The capture device of claim 14, wherein said ligand L is an antibody.

16. The capture device of claim 10, wherein said device further comprises an inlet and an outlet feeding mechanism.

17. The capture device of claim 16, wherein said device further comprises a collection vessel.

18. The device of claim 10, wherein said array of capillaries is wrapped in a helical configuration.

19. A capture instrument, comprising a capture device according to claim 10, and a fluid control system.

20. This set of claims can be elaborated once you decide what might be included in the complete instrument. We can also include specific chemistries for the capture system if there are preferred embodiments.

21. A method for the separation of biological particles, the method comprising:
contacting said particles with a capture capillary, the capillary comprising:
a capture system on the luminal surface having the formula
S-Q-(X)ₙ-C L-(Y)ₘ-L
wherein S is said luminal surface of the capillary tubing; Q is a chemical linkage between the surface and X; X is an affinity reagent; n is 1 or 0; CL is a cleavable linkage; Y is a linker or affinity reagent; m is 1 or 0; and L is a ligand capable of specifically binding a moiety present on a desired particle subset;
binding said desired particle subset to said ligand L;
releasing said desired particle subset by contacting with a cleavage reagent specific for said cleavable linker CL;
collecting said desired subset from said capture capillary.

22. The method according to claim 21, wherein said particles are cells.

23. The method according to claim 21, wherein said capture capillary further comprises a zone comprising a negative selection capture system to which said desired particle subset does not bind.

24. The method according to claim 21, wherein said cleavable ligand CL comprises an oligonucleotide and said cleavage reagent comprises a nuclease.

25. The method according to claim 21, wherein said ligand L recognizes a cell surface antigen.

26. The method according to claim 21, wherein said ligand L is an antibody.

27. A method for the separation of biological particles, the method comprising:
contacting said particles with a selectivity cassette of the formula
X′-CL-(Y)ₘ-L
applied said particles to a capture capillary, the capillary comprising:
a capture system on the luminal surface having the formula
S-Q-X″
wherein X′ and X″ are cognate members of a specific binding pair; S is said luminal surface of the capillary tubing; Q is a chemical linkage between the surface and X; CL is a cleavable linkage; Y is a linker or affinity reagent; m is 1 or 0; and L is a ligand capable of specifically binding a moiety present on a desired particle subset;
binding said desired particle subset to said ligand L;
releasing said desired particle subset by contacting with a cleavage reagent specific for said cleavable linker CL;
collecting said desired subset from said capture capillary.
